(19) United States
(12) Patent Application Publication
(10) Pub. No.: US 2003/0028228 A1
(43) Pub. Date: Feb. 6, 2003

(54) Treatment of collagen

(76) Inventor: Bruce J. Sand, Oak Park, CA (US)

Correspondence Address: Colin P. Abrahams, Suite 400, 5850 Canoga Avenue, Woodland Hills, CA, 91367, US

(21) Appl. No.: 10/229,762

(22) Filed: Aug. 28, 2002

Related U.S. Application Data
(63) Continuation-in-part of application No. 10/113,361, filed on Mar. 29, 2002.
(60) Provisional application No. 60/280,670, filed on Mar. 30, 2001.
(60) Provisional application No. 60/311,518, filed on Aug. 11, 2001.

(51) Int. Cl. A61N 5/067; A61B 18/18; A61B 19/00; A61N 1/00

(52) U.S. Cl. 607/89; 606/4; 606/17; 606/3; 606/13; 606/27; 606/34; 607/101; 128/898

(57) ABSTRACT

A system and method for the treatment of ocular collagen connective tissue comprises identifying a portion of the ocular collagen connective tissue having a connector portion which transitions into the ciliary muscle and the lens of an eye. A source of energy is then directed at at least one selected site along the portion of the connective tissue, the amount of energy being sufficient to cause longitudinal shrinkage in the length of connective tissue.

### CROSS REFERENCE TO RELATED APPLICATIONS

This application is a continuation-in-part of U.S. patent application Ser. No. 10/113,361 filed Mar. 29, 2002, which application claims the benefit of U.S. Provisional Applications Nos. 60/280,670 filed Mar. 30, 2001 and 60/311,518 filed Aug. 11, 2001, all of which are incorporated herein by reference in their entirety.

### FIELD AND BACKGROUND OF THE INVENTION

This invention relates to methods and apparatus for modulating the phase transition of collagen connective tissue thus causing the collagen fibers to contract or shrink in linear dimension. The more specific application of this process and system is directed to site of insertion of the collagenous ciliary muscle tendon of the eye.

The invention has particular application when used for the enhancement of accommodation and the reduction of resistance to aqueous outflow

The anatomical site of ocular collagen is the location of both the aqueous filtration system and the derivation of the tendinous insertion of the ciliary muscle. The filtration system is facilitated by the trabecular meshwork located in the angle at the periphery of the anterior chamber of the eye. The ciliary muscle is the dynamic origin of the focusing mechanism or accommodation of the eye.

The Functional Morphology of the Trabecular Meshwork

The angle in the anterior chamber referred to above is formed by the iris root, the connective tissue in front of the ciliary body, and the trabecular meshwork up to Schwalbe's line. This is shown in FIGS. 1 and 2. Posteriorly the sclera protrudes inward by forming the wide, wedge-like scleral spur where the anterior ciliary muscle tips end and most of the trabecular meshwork begins (so-called corneo-scleral portion). The inner part of the trabecular meshwork is fixed to the connective tissue in front of the ciliary muscle and to the iris root and is continuous posteriorly with the uvea (so-called uveal portion of the trabecular meshwork).

It has been concluded that the exact location of the resistance to aqueous outflow, thus affecting the intra-ocular pressure, is internally to Schlemm's canal in the trabecular meshwork.

Each lamella of the trabecular meshwork possesses a central core of densely packed collagen fibers running predominantly in an equatorial direction. The central core of the trabecular lamellae contains numerous collagen and elastic fibers embedded in a homogeneous ground substance.

Ciliary Muscle Tendons

It has been shown that the anterior ciliary muscle tendons are closely connected with the fiber network of the trabecular meshwork. There are three different types of tendons by which the anterior ciliary muscle tips are connected with the trabecular meshwork or the corneosclera.

Type I tendons derive from the outermost longitudinal muscle bundles and enter the sclera or the scleral spur to fix the muscle to the external tunica of the eyeball. Type II tendons pass the scleral spur to anchor within the trabecular meshwork. Type III tendons represent broad, elongated bands that penetrate the trabecular meshwork and insert within the corneal stroma. These tendons represent the main fixation of the entire ciliary muscle system to the external tunica of the eyeball and, therefore, are important to the accommodative mechanism. These tendons also help to expand the system of trabecular lamellae, so that the inter-trabecular spaces remain open or enlarge if the ciliary muscle moves forward and inward. Regarding the outflow resistance, this would have little effect in normal eyes. The proximity of the ciliary muscle tendons and the trabecular meshwork is illustrated by FIG. 2.

The main effect on aqueous outflow resistance seems to result from the actions of the elastic-like type I and II tendons. Since the type I tendons connect the outermost ciliary muscle fiber bundles to the scleral spur, muscle contraction leads to a backward movement of the scleral spur followed by a change in the form of the outflow pathways.

Inward movements of the type II tendons during muscle contraction have a similar effect. After ciliary muscle contraction, the cribiform elastic-like fiber network is pulled inwardly and the connecting fibrils are straightened so that the entire cribiform layer expands. In addition, the lumen of Schlemm's canal will be enlarged so that the filtering area increases and outflow resistance decreases. FIG. 3 shows this intimate relationship. The letter A represents the non-filtering portion of the trabecular meshwork; and the letter B shows the filtering portion of the trabecular meshwork comprising: the 1. iridial meshwork; the 2. uveal meshwork; the 3. corneoscleral meshwork; the 4. cribiform layer; and the 5. ciliary meshwork.

It has been known for a long time that the drug pilocarpine, reduces intra-ocular pressure. It has been shown that the outflow-resistance lowering effect of pilocarpine is exclusively due to ciliary muscle contraction.

This hypothesis is strongly substantiated by the disinsertion studies of researchers. If the anterior tendons of the ciliary muscle are cut so that the anterior ciliary muscle tips loose their contact with both the scleral spur and the trabecular meshwork, miotics lose most of their resistance-lowering effect.

Description of the Existing Technology

A classical theory of accommodation states that the relative diameter of the ciliary body in the steady state of the unaccommodated eye maintains constant tension upon a circular or circumferentially disposed assembly of many radially directed collagenous fibers, the zonules, which are attached at their inner ends to the lens capsule. The outer ends of the zonules are attached to the ciliary body, a muscular ring of tissue located just within the outer supporting structure of the eye, the sclera. This arrangement serves to maintain the lens at its minimal anterior-posterior dimension at the optical axis. The refractive or focusing power of the lens is thus relatively low and the eye is focused for clear vision of distance objects.

When the eye is intended to be focused upon a near object, the muscles of the ciliary body contract causing the ciliary body to move forward and inward, thereby relaxing the tension upon the zonules on the equator of the lens capsule. The inherent elasticity of the lens capsule and/or the lens itself permits a passive increase in the anterior-posterior dimension of the lens. The lens becomes more spherical resulting in an increase in the refractive or focusing power of the lens. This is the accommodative state of the lens.

According to the conventional view, as one ages, the lens becomes less malleable or the capsule less elastic and in spite of the reduced tension of the zonules upon the lens, the lens does not assume a greater curvature. The loss of elasticity of the lens and capsule is seen as irreversible. This is presbyopia.

Schachar has contributed a different theory regarding the cause of the loss of amplitude of accommodation that constitutes presbyopia. According to this view, accommodation in the non-presbyopic eye is not due to relaxation of the lens and capsule when the zonular tension is relaxed as a result of the contraction of the ciliary muscle. On the contrary, the contraction of the ciliary body exerts a tension on the zonular fibers that in turn actually results in an increase in the equatorial diameter of the lens and a corresponding increase in the central volume of the lens. These regional volume changes are responsible for the change in the optical power and accommodation of the lens. According to this theory, presbyopia results when the distance between the ciliary body and the equator of the lens and its capsule decreases with age as a result of the continued normal growth of the lens. Consequently, the radial distance between the equator of the lens and capsule and the ciliary body decreases throughout life.

Schachar claims that any method that increases the radial distance between the lens and ciliary body is effective in the method of his invention. He includes procedures that shorten the body of the ciliary muscle itself or move the insertions in the scleral spur and choroid, which can be employed to increase the effective working distance of the muscle.

Most of his disclosure is, however, directed to the weakening of the sclera. He does disclose methods for shortening the ciliary muscle itself by scarring it with various types of radiation. This also extends to scarring the adjacent tissue to accomplish this result. The effective working range may also be increased by moving the insertions of the muscle.

Schachar has disclosed methods for increasing the effective working distance of the ciliary muscle by increasing the radial distance between the equator of the crystalline lens and the inner diameter of the ciliary muscle by manipulating this muscle through external intervention. Schachar expands the sclera adjacent to the ciliary body in order to increase the effective working distance of the muscle. He further describes methods for repositioning the insertion of the ciliary muscle surgically or by applying heat directly upon the muscle or upon the adjacent tissue within the eye. The heat might be generated by ultrasonic or coherent energy. Reported complications of this procedure have been anterior segment ischemia and cosmetic blemishes.

Another scleral weakening process is described Dr. J. T. Lin. This process is called laser presbyopic correction (LPC). In this procedure, an erbium:YAG laser emitting at 2.93 u, sequentially ablates away scleral tissue until the choroid is visible through the overlying thinned scleral tissue over the ciliary body. This process is based upon the hypothesis that the sclera become more rigid with age thus attenuating the movement of the ciliary muscle. Laser ablation of this tissue in each quadrant (between the extraocular muscle insertions) would facilitate ciliary muscle action by weakening and invaginating the sclera, thus allowing the lens to change its shape and accommodate. A potential complication of this process is rupture of the globe.

Patents have been granted to Sand disclosing the method and apparatus for controlled linear contraction or shrinkage of collagen fibers to provide a multitude of non-destructive and beneficial structural changes and corrections within the human body. While this invention has application to the alteration of collagen connective tissue throughout the body, specific reference has been made to the correction of refractive disorders of the cornea of the eye.

Prior investigations have not considered the importance of the atraumatic attainment of the proper thermal profile for protracted or permanent collagen shrinkage. Consideration has not been given to the importance of maintaining the thermal profile in the target tissue within the thermal shrinkage temperature of collagen (Tₛ) of about 23 degrees Celsius above ambient body temperature plus or minus 4 to 5 degrees to stay below the traumatic threshold of the tissue. Maintaining this thermal profile prevents changes in the birefringence or optical axis rotation of crystalline collagen tissue. Exceeding the traumatic threshold will cause coagulation and scarring of the normally crystalline molecule thus precipitating replacement of the tissue and a wound repair cascade. Change in birefringence is, therefore, a marker for thermal damage in the tissue.

In the absence of trauma, the half-life of collagen has been shown to be consistent with the life of the experimental animal. Current developments have failed to take in to consideration that maintaining the proper thermal profile will prevent loss of the shrinkage effect as a function of time. It is therefore desirable to achieve controlled shrinkage of a collagenous matrix of tendinous tissue and thus increase its functional mechanical advantage in its effect upon the non-collagenous muscle. The present invention, in one aspect, addresses increasing the effective working distance or range of the non-collagenous muscle tissue by the atraumatic shrinkage of the collagenous tendon into which it inserts.

Dorlands's Illustrated Medical Dictionary defines a tendon as a “fibrous cord by which a muscle is attached.” Fibrous cord refers to the collagen connective tissue of which the tendon is constructed. The basic structural fiber in all connective tissues is collagen.

The bio-mechanics of a tendon substantially differentiates it from muscle tissue. It is important, therefore, to understand the mechanical response of collagen connective tissue in terms of its hierarchical structure as illustrated in FIG. 4. Beginning at the molecular level with tropocollagen, progressively larger and more complex structures are built up on the nano- and microscopic scales. At the most fundamental level is the tropocollagen helix. These molecules aggregate to form microfibrils which, in turn, are packed into a lattice structure forming a subfibril. The subfibrils are joined to form fibrils in which the characteristic 64 nm banding pattern is evident. It is these basic building blocks that, in the tendon, form a unit called a fascicle. At the fascicle level, the wavy nature of the collagen fibrils is evident. Two or three fascicles together form the structure referred to as a tendon. It is this multi-level organization that imparts toughness to the tendon. If the tendon is subjected to excessive stress, individual elements at different levels of the hierarchical structure can fail independently.

The tendon is subjected almost exclusively to uniaxial tensile stress oriented along its length. This situation requires that the tendon be elastomeric yet sufficiently stiff to efficiently transmit the force generated by the muscle. At the same time, it must be capable of absorbing large amounts of energy without fracturing. It accomplishes this through this unique hierarchical structure in which all the levels of organization from the molecular through the macroscopic are oriented to maximize the reversible and irreversible tensile properties in the longitudinal direction without fracture.

Collagen fibers in the tendon have a planar crimped geometry that is not present in muscular tissue. This fiber morphology is reflected in the shape of the stress-strain curve. The curve has three distinct regions corresponding to the state of deformation in the collagen fibers. These are a toe region of increasing modulus where the fiber crimp is gradually straightened, a region of constant modulus where collagen fibers are stretched elastically, and a yield region of decreasing modulus where fibers are irreversibly deformed and damaged.

This generality across species and tissue lines indicates the ubiquitousness of this crimp morphology and its importance in determining the mechanical response of all soft connective tissues, such as tendon.

The foregoing explains the increased mechanical advantage afforded the tendinous collagenous matrix following hydrothermal shrinkage imparted to the associated muscle without shortening of the ciliary muscle, without damaging or scarring of the muscle or adjacent tissue, and without moving or repositioning the muscular insertion.

Methods for reducing the resistance of the aqueous outflow in the treatment of chronic open angle glaucoma and ocular hypertension have been disclosed. Argon laser trabeculoplasty (ALT) has been advocated for this condition for over 20 years, and yet this procedure will aggravate existing glaucoma in 3 to 6% of the cases. It fails to arrest the progress of visual field deterioration in approximately 15% of these cases. Medical therapy must, therefore, be continued in these cases. Potential complications must also be considered. Among the more serious complications is inflammation manifested by iridocyclitis and peripheral anterior synecchiae or adhesions across the filtration angle. The greatest concern, however, is that the procedure may not be effective or that the glaucoma may become worse following the procedure. In fact, ALT has been shown to fail most commonly in the first year following the procedure in 23% of the cases.

Studies comparing the effectiveness of 810 nm diode laser trabeculoplasty and Q-switched frequency double Nd:YAG 532 nm lasers (SLT) have shown little advantages over conventional ALT.

### SUMMARY OF THE INVENTION

In one aspect, the invention is for a method and apparatus for the modulation of the phase transition of collagen connective tissue resulting in atraumatic shrinkage of collagenous matrix in the area of the scleral spur of the eye. In one application, this method is useful in the treatment of presbyopia. Accordingly, it is an aspect of this invention to provide apparatus and a method for the treatment of presbyopia.

A further aspect of the invention is to provide a method for treating presbyopia and/or hyperopia by shrinking the collagenous tendon of the ciliary muscle thereby increasing its functional mechanical advantage without shortening the muscle or moving its insertion.

A further aspect of the invention is to provide a method for increasing the range and amplitude of accommodation of the eye.

A further aspect of the invention is to provide a method for the facilitation of accommodation in the replacement of the natural crystalline lens with the intracapsular implantation of an accommodating intraocular lens.

Still a further aspect of the invention is to provide a method for the reduction of the resistance to aqueous outflow in the treatment of chronic open angle glaucoma and ocular hypertension.

### BRIEF DESCRIPTION OF THE DRAWINGS

FIG. 1 shows an enlarged view of the anterior chamber angle of the eye;

FIG. 2 shows the anatomical site of the ciliary muscle and its tendon in its relationship to the aqueous filtration system of the eye;

FIG. 3 shows a schematic view of the anterior chamber angle and the relationship of the ciliary muscle to the filtration system of the eye;

FIG. 4 shows the hierarchical structure of the tendon thus differentiating it from muscle;

FIG. 5 shows the change of geometry of the anterior segment from the unaccommodated (left) to the accommodated state (right), as illustrated in Hehnholtz's Treatise on Physiological Optics;

FIGS. 6(a) and 6(b) show Rohen's schematic representation of accommodation mechanism;

FIG. 7 shows the schematic representation of zonular geometry based on the studies of Famsworth and Burke;

FIG. 8 shows a schematic representation of the hydraulic suspension model of Coleman;

FIG. 9 is a plot of the absorption coefficient of water (the universal chromophore) as a function of incident wavelength; and

FIG. 10 shows a laser delivery system with integrated passive heat sink in accordance with one aspect of the invention.

### DETAILED DESCRIPTION OF THE INVENTION

Accommodation and Presbyopia

The accommodative state of the crystalline lens is the result of the action of the ciliary musculature. The exact mechanism is poorly understood but one thing is not controversial: all of the fibers of the muscle, irrespective of site, will get thicker during contraction. The effect of this will be to increase the cross-sectional diameter of the whole muscle and make the border of the muscle move inwards towards the inner edge of the ciliary body. Thus the whole muscle, including the longitudinal fibers, will in effect act like a sphincter to the ciliary ring. In this connection, it is noted that the ciliary muscle is thickest approximately opposite the equator of the lens. Contraction of the ciliary muscle has an effect upon accommodation.

In any case, shortening of the muscle in a longitudinal sense by means of contraction or shrinking of a parallel segment of the muscle or its direct insertion will increase the mechanical advantage of the muscle and augment its action. This will result in the enhancement of the accommodative range and amplitude of the lens. In a collateral sense, this action at the scleral spur insertion of the muscle will increase the pore size of the aqueous filtering trabecular meshwork and thus reduce intra-ocular pressure, as well.

Accommodation is the process by which the overall refractive power of an eye is altered to allow focus of an image upon the retina of the eye. Humans appear to have their own unique solution to the problem of achieving an extensive focusing range, which differs from the remainder of the animal kingdom. It involves carefully controlling the changes in the shape and thickness of the lens within the eye.

When the eye is focused on infinity (about 20 feet and farther), the crystalline lens is at its flattest and thinnest relative to the optical axis. For the eye to focus closer than this, the ciliary muscle contracts, the degree of contraction being correlated with the increased sharpness of the lens curvature and increased lens thickness along the optical axis. Since the lens and ciliary muscle are only indirectly attached, through the zonular (or suspensory) apparatus, the major question concerning accommodation at this time is the mechanism by which the ciliary muscle contraction and the lens deformation are coupled. There is, however, universal agreement that muscle contraction is the necessary ingredient for accommodation to occur.

Another issue related to accommodation and shared by all primates is the fact that the range of accommodative amplitude decreases with age, such that the nearest point that can be focused gradually recedes. This results in the need for optical prostheses for close work such as reading and, eventually, even for focus in the middle distance. The loss of near focus is actually progressive over a person's lifetime, irrespective of whether he or she is emmetropic, myopic (nearsighted), or hyperopic (farsighted).

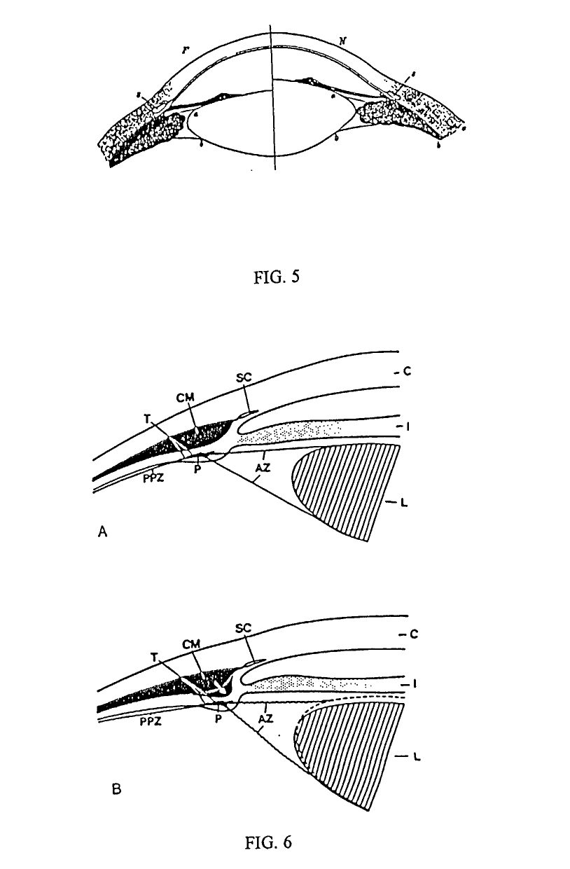

Although a number of hypotheses about the human focusing mechanism have been brought forward, the best known and most enduring is that of Hermann von Helmholtz in his Treatise on Physiologic Optics. His theory is illustrated by FIG. 5, in which the change in geometry of the anterior segment of the eye from the unaccommodated to the accommodated state reveals that the anterior chamber shallows due entirely to the change in shape and thickness of the lens. The center of mass is moved forward while the distance from the cornea to the posterior lens surface remains unchanged.

Modern versions of the Hehnholtz-Gullstrand mechanism for accommodation are in agreement that the process involves the direct action of the ciliary muscle contraction upon the lens and that upon contraction, the net mass of the ciliary muscle moves anteriorly and inward, the latter resulting in a reduced inside diameter.

Rohen's representation of accommodation is shown in FIGS. 6(a) and 6(b) of the drawings, in which the zonules attach to the ciliary body at a point that acts like a pivot or fulcrum during muscle contraction, such that this point is moved forward and inward in accommodation. The anterior zonules (AZ) are completely relaxed, while the orientation of the posterior zonules (PPZ) is altered consistent with the increased posterior lens sharpness of curvature.

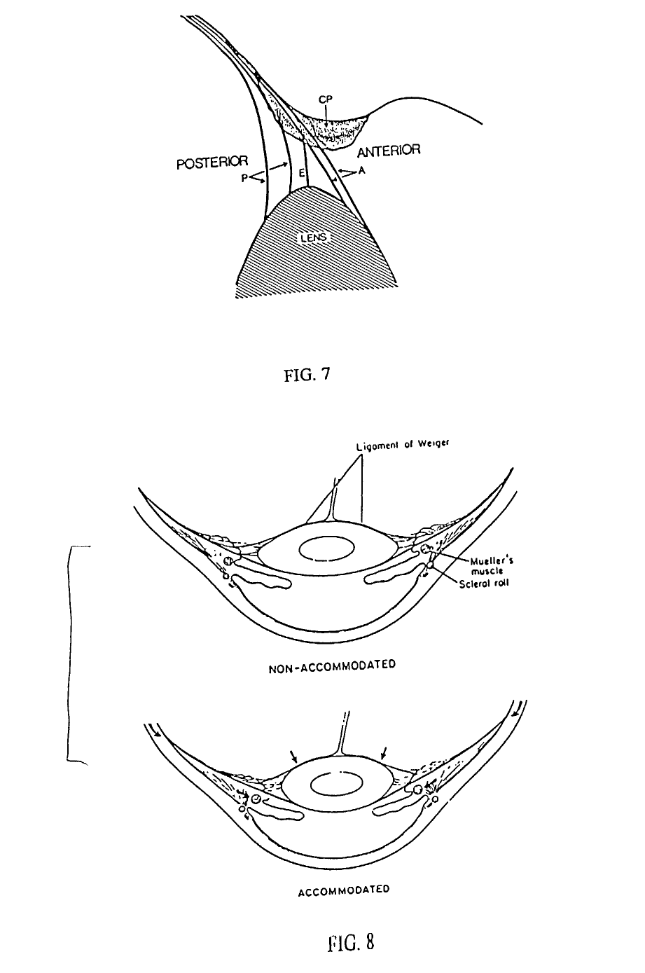

The zonular apparatus geometry based upon the studies of Famsworth and Burke is represented by the schematic in FIG. 7. In contrast to Rohen's model, the attachment of the anterior and posterior zonules (A and P in FIG. 7) to the ciliary body is posterior to their attachment to the lens capsule. The contraction of the ciliary muscle will result in a more complex relaxation of the tension on the lens.

D. Jackson Coleman created another explanation for accommodation as shown in FIG. 8, in which contraction of the ciliary muscle results in a small pressure increase in the vitreous, which is sustained during accommodation. Fisher, in 1977, put forth another theory that ciliary muscle force, combined with the elastic molding properties of the lens capsule, was sufficient to account for accommodation.

It seems clear that there is good qualitative agreement as to the events occurring during accommodation, but serious disagreement over the role of lens-associated structures in the process. Until these points have been resolved, the question of the “true mechanism” of accommodation in the human eye will remain a matter of personal preference. Whatever the model of accommodation, there is no disagreement that the contraction of the ciliary muscle plays a central role, affecting the lens either directly through the zonular apparatus and capsule, indirectly through a vitreal hydraulic force, or through some combination thereof. Thus, the ways in which the ciliary muscle and associated tissue age become of paramount importance.

Rohen and Lutjen-Drecoll et al, who studied the aging in ciliary muscle specimens, discovered that the ciliary muscle exhibits age-related structural changes (e.g., increasing numbers of lysosomes, degeneration of some muscle cells, etc.) and loss of pharmacologic sensitivity to pilocarpine on a time-scale related to accommodative amplitude loss; this time-scale suggests a degenerative change in muscle structure and function at a young adult (16 to 20 years) age. In addition, the location of the internal apical region of the ciliary body in humans is moved forward and inward with aging, suggesting that the tension exerted by the zonular apparatus upon the lens may be decreasing. All of this data are of significance in suggesting the importance of ciliary muscle ultrastructural and functional investigations in the human.

For those models that postulate a direct link between ciliary muscle contraction and change in lens shape, an alteration in the properties of either the muscle, zonules, or the lens could lead to a loss of accommodative range. Thus, degradation of the muscle's contractile ability, and/or changes in the three dimensional geometry of the ciliary muscle-zonule-lens system would affect the accommodation process. If the muscle is reduced in contractile power over time, all other factors being unaffected, this would directly affect the accommodative range, since the degree of lens elastic recovery is directly linked to the degree of muscle contraction. Alternatively, muscle contraction might not be reduced with age, but its excursion as part of the accommodative mechanism might be; this hypothesis is still in the process of development, but it suggests that the effective result of this loss would be a reduction in the degree to which the lens is allowed to accommodate.

This invention discloses methods and apparatus for the controlled thermal phase transition resulting in the shrinkage of collagen connective tissue at the site occupied by both the tendinous insertion of the ciliary muscle and the trabecular meshwork of the aqueous filtration mechanism.

Previously, there has been no practical method of enhancing the mechanical advantage of skeletal or non-skeletal musculature. Studies of the effects of thermal shrinking of collagen connective tissue has, however, led to other clinical processes, such as laser thermal keratoplasty (LTK) for the treatment of refractive errors, treatment of herniated discs by thermal shrinkage of annulus fibrosis, treatment of unidirectional and multidirectional glenohumeral instability by means of laser-assisted capsular shift, ligament shortening in medial collateral ligament laxity in the knee by laser induced thermal shrinkage, laser-induced anterior cruciate ligament shortening for unstable joint disease, and shortening of extra-ocular muscle tendon in strabismus by laser thermal contraction known as thermal tendinoplasty.

In each case, the non-ablative application of infrared laser energy increases the temperature of the collagenous matrix to the thermal shrinkage temperature of collagen (Tₛ), which is about 23 degrees Celsius above ambient body temperature but below that temperature of coagulation and tissue destruction. It has been known for over 100 years that collagen contracts to ⅓ of its lineal dimension immediately upon reaching that temperature.

One indisputable fact, however, remains. That is, irrespective of the theory presented, contraction of the ciliary muscle is required for accommodation to occur, and age-related structural changes in presbyopia directly alter its ability to efficaciously maintain appropriate range and amplitude of accommodation.

One cannot strengthen the muscle, but shortening (by means of laser-induced thermal shrinkage) of the collagenous muscle tendon will directly affect the mechanical contractile effect of the muscle.

Intraoperative observations of intracapsular cataract extractions have revealed that most cataractous lenses are malleable enough to deliver through relatively restrictive corneal-scleral incisions, irrespective of age. Similarly, phacoemulsification techniques do not require that the emulsification energy be altered except for the more mature cataracts.

This having been stated, the process herein disclosed for increasing the apparent mechanical advantage of the ciliary musculature might have more specific applicability for the younger presbyope whose lens still retains residual malleability.

As stated earlier, the accommodative function is complex and multifactorial. The contraction of the ciliary muscle causes the net mass of the ciliary body to move forward or anteriorly, as well as inward. This forward movement also serves to increase the range and amplitude of accommodation. The older presbyopic individual, therefore, will still experience some improvement in focusing ability, in spite of the loss of lens elasticity and reshaping capability.

Microscopic Anatomy

A review of the anatomy and the histology of the ciliary musculature is key to understanding these cause-effect relationships.

The ciliary muscle has always been considered to have three portions; meridional, radial, and circular. Some believe that there is little justification for dividing the muscle into three parts. The whole muscle is interconnected, the muscle bundles forming a three-dimensional reticulum with considerable interweaving of the muscle cells from layer to layer. It is believed that the entire ciliary muscle originates from the scleral spur region and inserts into the iris, the ciliary processes, and the choroid. Calasans described the muscle as arising from the ciliary tendon, which includes the scleral spur and adjacent connective tissue. The muscle bundles of the longitudinal, radial, and circular portions are oriented in the ciliary body in a certain pattern only because of their method of origin from the scleral spur and the direction of their muscle cells.

The ciliary tendon gives rise to the many paired V-shaped bundles of the longitudinal muscle. The base of the V is at the scleral spur and its apex is in the choroid. The bundles of the longitudinal portion lie in the outer part of the ciliary body and they end in the so-called epichoroidal muscle stars in the anterior third of the choroid. This epichoroidal attachment anchors it somewhat to the sclera and there is considerable interweaving of the V-shaped bundles with each other. Internal to the longitudinal muscle bundles is another group of bundles, the radial or oblique portion of the ciliary muscle. These interweaving and crossing cells also arise as paired V-shaped groups from the ciliary tendon. All of these V-shaped bundles insert by tendinous processes into the connective tissue of the anterior or posterior portion of the ciliary processes, depending on their origin from the scleral spur. The two arms of the V-shaped bands, which form the circular muscle bundles, arise from very wide attachments to the ciliary tendon and insert into the connective tissue in the region of the anterior ends of the ciliary processes. Additional muscle bundles, the iridic portion, arise from the most internal region of the ciliary tendon as a pair of arms that are also united into a V. They form two thin tendinous processes which insert into the iris near the termination of the dilator muscle.

The connective tissue separating the muscle bundles is thin and compact in the longitudinal portion and dense and thicker in the radial portion, so that it produces a greater separation of the muscle bundles.

The anterior extension of the ciliary muscle and its relationship to the trabecular meshwork has been studied extensively. Part of the longitudinal portion of the muscle can be traced to the scleral spur, where the tendons pass through the spur into the posterior corneo-scleral trabecular meshwork. There may be a continuity between the muscle and the meshwork and, except for the portion adjacent to Schlemm's canal, most of the meshwork represents a tendon of the meridional muscle that inserts into the cornea at Schwalbe's ring.

The smooth muscle cells are surrounded by a thin sheath of fibroblasts and are separated from each other by collagen, blood vessels and fibroblasts.

Mechanism of Action

The invention involves the technique required to shorten or shrink the tendinous portions of the ciliary musculature in order to increase its mechanical advantage. This mechanism is necessary to overcome the physiologic laxity in the accommodative function brought about by the onset of presbyopia.

Shrinkage at this site will, as a collateral action, effectively pull the trabecular meshwork open thus increasing the pore size and reducing intra-ocular pressure. The laser exposure would also reduce the circumference of the trabecular ring by heat-induced shrinkage of the collagenous trabecular sheets forcing the ring centrally. This will effectively elevate the trabecular sheets and pull open the inter-trabecular spaces, thereby reducing resistance to outflow.

Technology has been disclosed in the prior art by which coherent energy in the appropriate wavelength domain has been utilized to contract or shrink collagen connective tissue causing nominal trauma to the tissues of regard. Infrared laser energy, both pulsed and continuous wave, has been selected by means of extinction depth or its reciprocal, spectral absorption coefficient, to match the desired histological depth of the tissues of regard. For example, mid-infrared laser energy emitting a wavelength of approximately 2 microns is absorbed at approximately 350 micron depth in water. This depth coincidentally matches the thickness of the water-containing mid-anterior stroma of the cornea. This results in an absorbed thermal profile, which is appropriate for the shrinking of and recurvature of the cornea of the eye, a process called laser thermal keratoplasty (LTK).

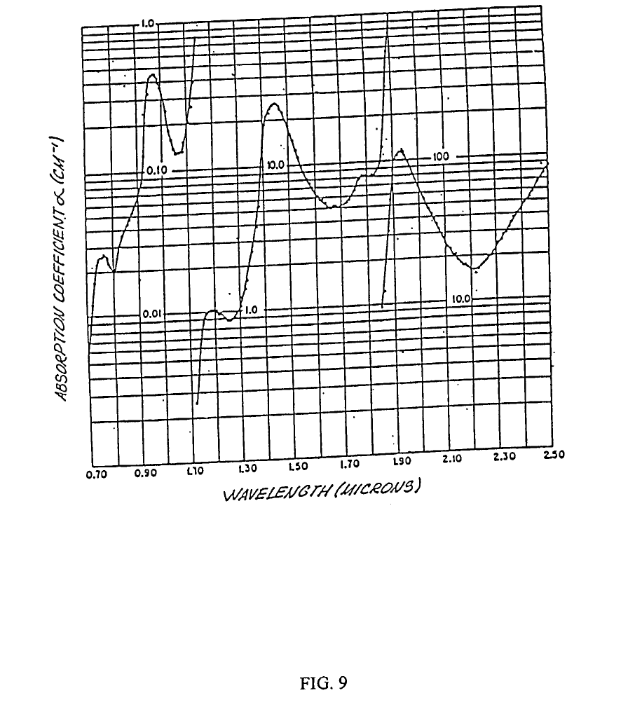

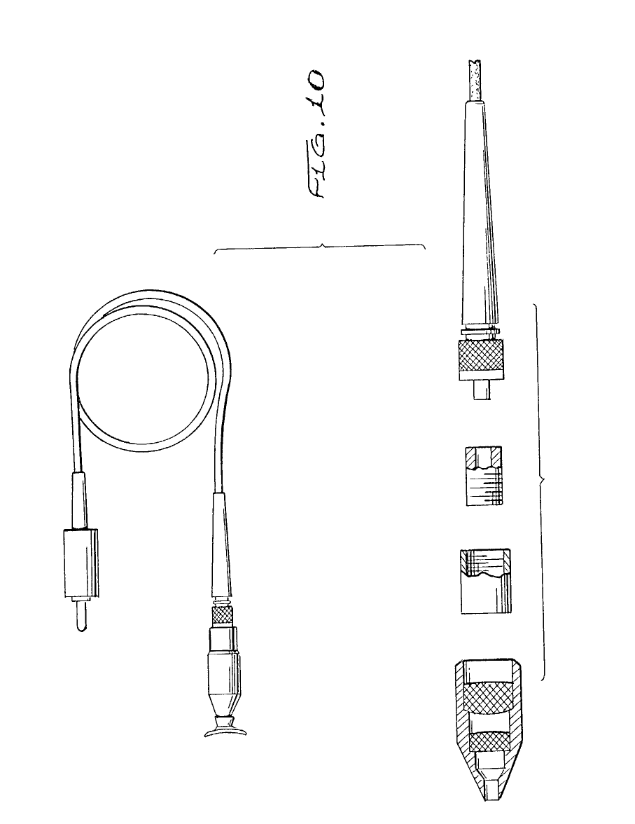

Utilizing this concept, the present invention discloses the selection of an infrared laser emitting in the wavelength of 1.32 microns with an extinction depth of about 800 to 1000 microns. This depth of absorption matches the histological depth of the water-containing collagenous matrix of the ciliary muscle tendon, as shown in FIG. 9. The wavelength dependency of this variable has been previously disclosed, and this figure is a textbook graph plotting absorption coefficient (water) against wavelength. While solid-state diode lasers might be fabricated to emit at this wavelength, a pulsed Neodynium:YAG laser, which can be operated at a repetition rate of from 1 Hz to 100Hz is commercially available. The laser operates within an energy range appropriate for causing hydrothermal shrinkage of collagen. Recently, however, a diode array solid-state laser system emitting in the same wavelength has become available. This continuous wave laser may generate a preferable thermal profile.

In a further utilization of the concept, the present invention discloses the selection of lasers emitting in the wavelength range from 500 um to 11000 um. An example of the appropriate wavelength laser might be one which emits irradiation in the visible spectrum at about 532 um. While this laser wavelength has been used extensively for trabeculoplasty, the method of using this laser for the expressed indication of shrinking collagen in either the core of the trabecular lamellae or the scleral spur has not been previously disclosed. Another laser wavelength, which might under the proper conditions be used for the disclosed method, might be 1.32 microns. Other lasers might be efficacious, such as 1.66 microns or 1.73 microns.

There is an interesting scleral window for 1.73 um. The following solid state lasers that will operate in that region are: 1. Erbium doped: YLF @ 1.73 um, and 2. Erbium doped: YAP @ 1.66 um

Water absorption is moderate at these wavelengths; this certainly dominates the photothermal axial transmission with minimal scatter; this should permit diode pumping to obtain a CW emission. The lasing rods are being fabricated to sustain 2 to 5 Watts delivered via fiber. Since scatter would be minimal, surface cooling will be much easier. This wavelength will have good AxT in spite of oblique angle of incidence focused about X2 to paint tissue without photocoagulation.

While pulsed lasers might obtain the appropriate thermal profile in the target tissues, those which irradiate with a continuous wave emission might be more efficacious. This would include pulsed lasers operating with protracted pulse wave duration or rapid repetition rate, thus simulating a continuous wave emission. Diode pumped lasers and diode array lasers may also be appropriate.

It would be preferable to direct this coherent energy by means of a trans-scleral route directly to the collagenous tendinous insertion of the ciliary muscle. It would be even more preferable to direct this energy to the area of the scleral spur wherein the base of the V-shaped bundles of longitudinal ciliary muscle originates from the ciliary tendon. This energy can be easily directed under direct visualization to the scleral spur without the risk of damage to other important structures, such as the crystalline lens or the ciliary body.

It might also be possible to choose the appropriate laser wavelengths and reflecting optics to direct the coherent energy across the cornea and anterior chamber of the eye to the scleral spur ab interna. This route is called the gonioscopic approach. In this case, one must select laser wavelengths in which the energy is absorbed deep enough in the target tissue to be effective and have little water, melanin and hemoglobin absorption characteristics.

While methods disclosing the use of coherent energy are presented, this preferred method is not meant to exclude other types of energy sources, such as microwave, radio frequency, ultrasonic, etc. For example, radio frequency energy generates elevated temperature in the target tissue by the utilization of low energy, high frequency (radio frequency, 350 kHz) current. In this way, collagen within the treatment zone is heated in a gentle, controlled fashion as a result of the natural resistance the tissue to the flow of current.

Damage to the lens might result in cataract formation. Two safety factors avoid this concern. The 1.32 micron emission is strongly absorbed by water. Any energy, which might penetrate beyond the target tissue would be immediately extinguished before causing an elevation in temperature of the aqueous humor sufficient enough to cause lens damage. Additionally, the present invention describes a direct contact delivery system, which under direct visualization will direct the infrared energy to the target scleral spur. The iris root further protects access to the lens by the laser.

Damage to the ciliary body might cause inflammation and aqueous hyposecretion. The use of the selective trans-scleral delivery system prevents application of thermal energy to this area in the posterior chamber of the eye.

Laser Delivery System

The contact laser delivery system consists of a 200 or 320 micron diameter quartz fiber-optic probe housed in a protective casing giving a total outer diameter equivalent to a 22 gauge needle. The tip of this fiber-optic may be fabricated so that the energy is transmitted at approximately 90 degrees to the fiber axis with a posterior coating of gold thus preventing back scatter of the energy. Another embodiment of the delivery probe might be a straight hand-piece into which the fiber-optic cable is inserted for the ease of handling during delivery of the energy to the eye. This is illustrated in FIG. 10 of the drawings. Other variations of this delivery system might be advantageous.

A Helium Neon laser aiming beam is directed along the probe for easy identification of the operative site, since the infrared laser emits an invisible wavelength of light.

The Procedure

Diagnostic gonioscopy of the filtration angle structure is mandatory in all eyes prior to surgery. A Goldmann 3-mirror gonioprism is recommended for high quality viewing of the structures, although a Goldmann single mirror lens may be used. One should identify Schwalbe's ring and the scleral spur, which is the target site for the laser energy. Energy will be applied in all four quadrants of the globe in order to shrink the ciliary muscle tendon equally. In many cases, the scleral spur may be difficult to visualize in all quadrants due to pigment. In this case, the patient is requested to look in the direction of the examination mirror and the fixation light should be repositioned in the same direction as the mirror.

The process of photothermal shrinking of the ciliary muscle tendon at the site of the scleral spur for the enhancement of accommodation is accomplished at the slit lamp.

In the employment of the slit lamp for focal examination of the visualizing of the filtration angle of the eye, six methods are available. Diffuse illumination, direct illumination, retroillumination, specular reflection, indirect lateral illumination and oscillatory illumination may each be employed depending upon the choice of the detail desired.

A novel method, not previously described, has significant advantage over the other methods. Staining the corneal and bulbar conjunctiva with fluorescein dye in an alkaline 2% solution is valuable in delineating the corneal-scleral trabecular meshwork, which might not be visible by any of the previously described methods of biomicroscopic illumination, alone.

While other dyes may be used, fluorescein dye is the most effective. A suitable formula for the dye is as follows:

| | | |
|---|---|---|
| Fluorescein sodium | 2.0 | parts |
| Phenylmercuric nitrate | 0.004 | (for sterility) |
| Distilled water | 100.00 | |

A topical anesthetic is instilled into the conjunctival sac followed by the dye. Sterile solutions combining both anesthetic and fluorescein are commercially available. The anesthetic enhances absorption of the dye through the intact cornea

After the dye has been instilled into the conjunctival sac, the lids are closed thus distributing the dye evenly over the surface of the eye resulting in a bright green layer. The dye is allowed to remain in the conjunctival sac for a few minutes behind the dosed lids instead of being washed out immediately. It thus penetrates the intact epithelium. The dye eventually reaches the anterior chamber where it is cleared by the filtration meshwork. The trabecular meshwork is thus stained a brilliant green as the normally orange fluorescein dye is excited by the cobalt blue filtered retro-illumination of the slit lamp.

The slit lamp is now employed to further localize the site of the scleral spur insertion of the ciliary tendon. The normally elusive meshwork has been rendered, thereby, visible. The site slightly posterior to the uveal meshwork and Schlemm's canal is then selected for irradiation as the red HeNe illumination is directed slightly posterior and oblique to the perpendicular. The corneal-scleral trabecular meshwork is 1½ mm wide as it is disposed circumferentially within the angle of the anterior chamber between the anterior placed Schwalbe's ring and the posterior limitation of the scleral spur.

While the slit lamp may be employed for the procedure, another method might be employed. The use of an integrated laser delivery system and contact cooling device might be utilized. This might be in the form of a handheld instrument.

Surface cooling confines the thermal profile at the appropriate depth, as described in more detail below.

The foot pedal of the laser is depressed enabling the 1.32 micron Nd:YAG infrared laser system operating at 300 microseconds pulse duration with a repetition rate of 3 to 20 Hz and a power setting of 1 to 6 watts. Energy per pulse of 6 Joules is obtainable and exposures of 3 pulses to CW are possible. The 0.5 mWatt HeNe 632.8 nm aiming laser transmits through the same optical pathway.

Reduction in Resistance to Aqueous Outflow in Glaucoma and Ocular Hypertension

The process employed for the reduction of resistance to aqueous outflow for chronic open angle glaucoma or ocular hypertension is essentially the same as that utilized to enhance accommodation.

The differences are related to the number of laser irradiation applications disposed circumferentially over the target sites. Furthermore, treatment for enhancement of accommodation would have little effect upon the resistance to outflow in normal eyes.

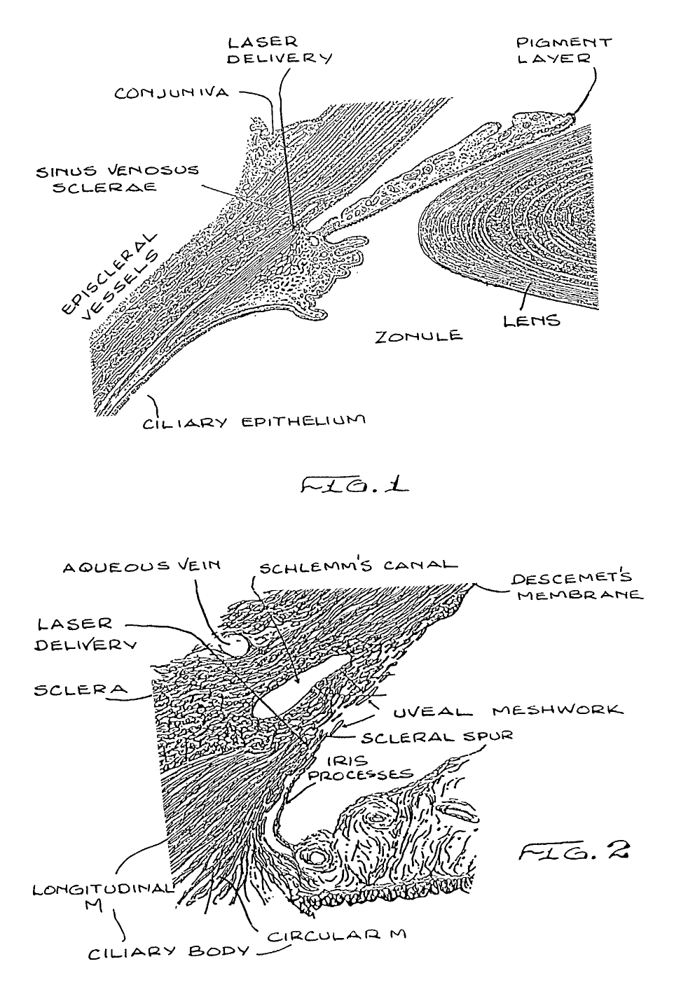

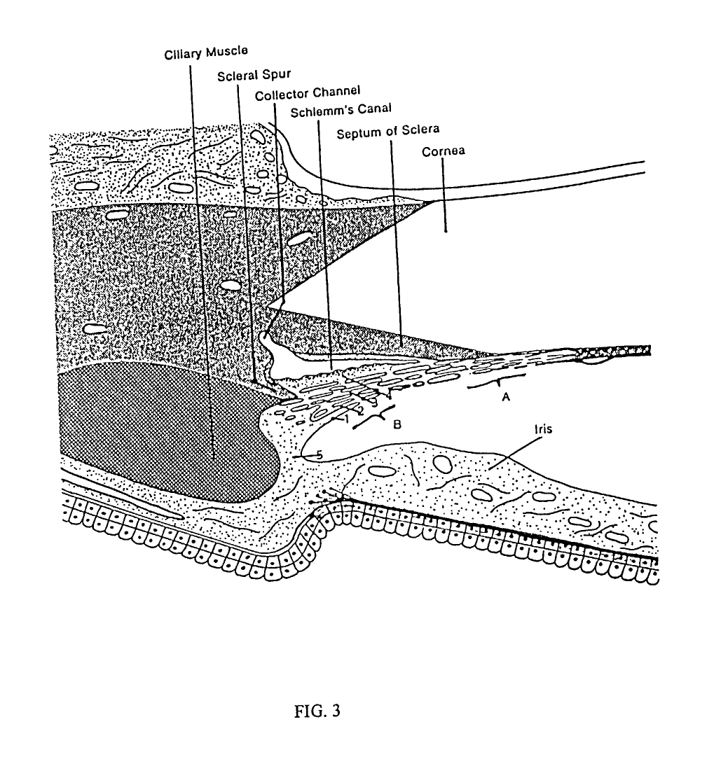

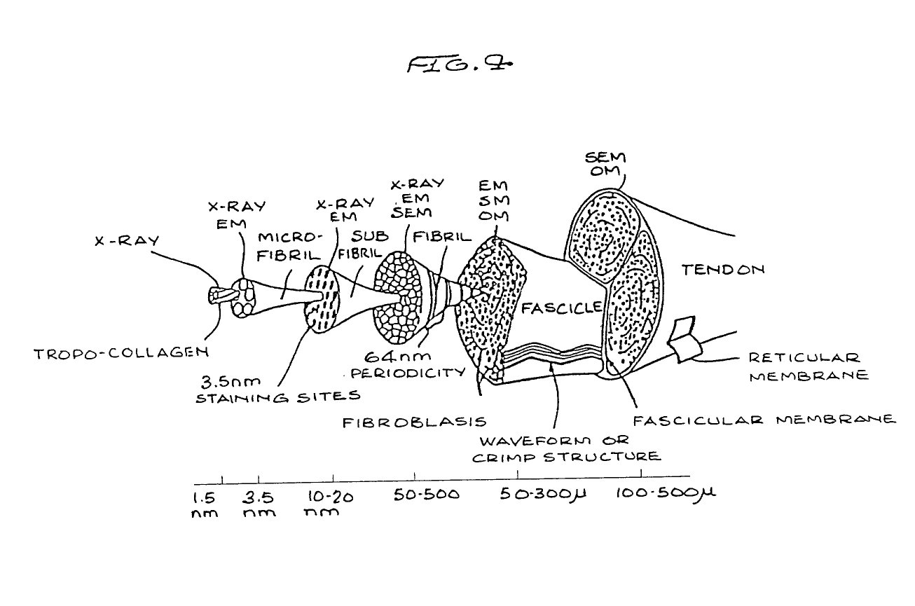

There are two explanations for the efficacy of this procedure, which set it aside from the prior art. One explanation is based upon the hypothesis that enhancement of the action of the ciliary muscle tendon from photothermal shrinking of the collagenous fibers results in the pulling inward of the entire cribiform network. The cribiform layer expands and the lumen of Sclemm's canal is enlarged so that the filtering area increases and outflow resistance decreases. Kaufman and Barany have substantiated this theory and the anatomical location of the target site is shown in FIGS. 1 and 2.

A second theory is grounded in the histo-pathology of the trabecular ring. A retrospective analysis assessing the efficacy of ALT has resulted in the following explanation: The laser exposures reduce the circumference of the trabecular ring by heat-induced shrinkage of the collagen of the sheets, or by scar tissue contraction at the argon laser burn sites. This then forces the ring toward the center of the anterior chamber thus elevating the sheets and pulling open the inter-trabecular spaces, thereby reducing the resistance to outflow.

The circumference of the trabecular meshwork is approximately 36,000 microns. One hundred argon laser burns of 50 microns each would involve 5000 microns of the meshwork, about 14% of the circumference, leaving 86% undamaged. If each burn had only a 5% shrinkage in diameter, this would reduce the trabecular circumference by 250 microns and the ring diameter by about 80 microns, thus elevating the trabeculum about 40 microns on each side. Even at its thickest point, the trabeculum has only 15 to 20 layers, so that the average increase per single inter trabecular space may be 2 microns or more. The normal inter-trabecular spaces have been estimated at 0.5 microns. A 2 micron increase would represent a five-fold increase in the gap available for aqueous flow between the trabecular sheets. Using these dimensions, even a 1% shrinkage from the laser burns might give a 50 to 100% increase in the inter-trabecular spaces.

The trans-scleral approach to the trabecular ring using nominal collagen shrinking energy of a mid-infrared coherent energy source appropriately selected for its spectral absorption characteristics is the desired method. Little or no trauma is sustained by this methods and, thus, there will no biological wound repair response generated.

The 1.32 micron Nd:YAG or 1.34 micron Nd:YAP lasers are each appropriate sources of coherent energy with an extinction depth near the depth of the target tissue as noted in FIG. 9. Both can be delivered by means of a fiber optic delivery system. Very precise methods of controlling the laser systems and optically filtering the produced light energy currently exist. By means of selection of the appropriate combination of resonance optics and/or anti-reflective coatings, wavelength in this range can be produced from the laser normally emitting in the range 1064 nm.

The 1.32 micron Nd:YAG or 1:34 micron Nd:YAP lasers are each appropriate sources of coherent energy. Other wavelengths might also be appropriate and depend upon laser beam propagation and transmission.

An appropriate laser system for this application might be the 1.32 micron Nd:YAQ laser-operating at 300 microsecond pulse duration with a repetition rate of 3 to 20 Hz and power of from 1 to 6 watts, such as that manufactured by New Star Lasers, Inc. of Roseville, Calif. Energy/pulse of 6 Joules are obtainable and exposures of 3 pulses to continuous wave are possible. An aiming beam from a 0.5 mW Helium Neon (HeNe) 632.8 nm laser might be integrated into the delivery system.

An additional embodiment might employ the use of a diode array solid state laser emitting in a continuous wave at 1.32 u. The advantages of the CW laser might be the lower risk of tissue ablation due to the lack of peak intensities and peak radiant exposures. CW radiation offers the possibility of a more homogeneous thermal profile within the tissue.

The thermal effect obtained from such a system is independent of tissue pigment absorption. The high absorption of this laser energy by the aqueous humor in the anterior chamber of the eye renders the energy impotent and self-extinguishing beyond the trabecular meshwork. This then obviates the potential sequellae observed with argon laser trabeculoplasty (ALT), such as iridocyclitis and transient elevated intra-ocular pressure. The traumatic wound healing response usually observed with ALT will not be experienced with this procedure. The actual trauma to the collagen will be nominal and consist only of a phase transition. Non-traumatized metabolically inert collagen is not normally replaced as a result of its long half-life. The pressure lowering effects, therefore, should be long lasting, if not permanent.

A clinically successful model for the use of mid-infrared coherent radiation for collagen shrinkage has been developed by the inventor. Sunrise Technologies International Inc. (Fremont, Calif.) employs the use of a Holmium:YAG pulsed laser operating at a wave length of 2.12 u for the simultaneous application of eight to sixteen laser spots upon the cornea in a radial pattern centered on the entrance pupil at an optical zone of 6.5 to 7.5 mm for correction of refractive errors. This specific wave length was selected to match the absorption depth of the laser to the depth of the target tissue. In this manner, an optimum thermal profile is obtained at the proper depth within the tissue to reach the thermal shrinkage temperature of collagen connective tissue (Tₛ).

The ab externo laser application procedure would be performed at the slit lamp with the patient in the familiar sitting position utilizing surface cooling and a specially designed quartz fiber optic delivery system. While normal office based sterile techniques would be recommended, a non-sterile environment would be acceptable since the procedure is non-interventional.

The corneo-scleral trabecular band is 1½ mm wide as it is disposed circumferentially within the angle of the anterior chamber between the anteriorly placed Schwalbe's ring and the posterior limitation of the scleral spur.

A drop or two of topical ophthalmic anesthetic, such as Ophthaine, is instilled into the conjunctival cul-de-sac. The patient is seated comfortably in front of the slit lamp with his chin on the chin rest and forehead against the head-rest. Diagnostic gonioscopy of the filtration angle structures to familiarize one with the anatomy is mandatory prior to the laser procedure. A Goldmann 3-mirror gonioprism is recommended for high quality viewing although a Goldmann single mirror lens may be used.

Staining of the cornea and bulbar conjunctiva with a suitable dye is a valuable method of demonstrating the extent of a disease process and a variation of this method is utilized to identify the target tissue for laser trabeculoplasty. Instilling fluorescein dye in a 2% alkaline solution is especially valuable in delineating the corneo-scleral trabecular meshwork. Sterile solutions combining both the anesthetic and dye are commercially available and the anesthetic enhances penetration of the dye into the anterior chamber through the intact cornea.

While other dyes may be used, fluorescein is the most effective. After the dye has been instilled, the lids are closed distributing the dye over the entire ocular surface. The dye eventually reaches the anterior chamber where it is cleared by the filtration mechanism. The trabecular mechanism is thus stained a brilliant green as the dye is excited by the cobalt blue filtered light from the slit lamp. Retro-illumination is then used to visualize the normally illusive target tissue through the slit lamp.

Surface cooling confines the appropriate thermal profile for collagen shrinkage to the target tissue.

In clinical practice, this method of reducing the resistance to aqueous outflow in chronic open angle glaucoma or ocular hypertension would be applied ab externo through the full-thickness conjunctiva and sclera. Approximately 50 laser applications would be applied over 180 degrees of the trabecular meshwork.

The procedure utilizing an infrared laser system emitting 1.32 micron radiation is advantageous. This laser has an preferable absorption depth of 800 to 900 microns thus matching the anatomical depth of the ocular trabecular filtration meshwork. This laser is commercially available and can be operated in the multi-pulse mode thus permitting closed loop monitoring of the laser-tissue thermal interaction by means of PPTR (pulsed photothermal radiometry). An alternative technique utilizes a solid-state diode CW laser system at the same wavelength.

This preferred thermal process is a photobiologic process utilizing coherent energy in the infrared wavelength domain. This invention also includes the use of other thermal processes, such as microwave and radio-frequency technologies for collagen shrinkage.

Photobiologic Basis for Invention

The advantage of laser light in the treatment of various types of tissues is that its monochromatic, high energy beam can be focused and manipulated to obtain specific photobiologic effects. Irradiation exposure parameters can be matched to specific physical, chemical, and biological properties of the target tissues to obtain a desired result.

Tissues may be defined by their (1) optical properties (absorption, scattering, and scattering anisotropy), (2) thermal properties (heat capacity and heat diffusivity), (3) mechanical properties (viscoelasticity, tensile strength and rupture points), (4) chemical composition (water and other endogenous and exogenous absorbers), (5) anatomy (physical arrangement of organelles, cells, and tissues), and (6) physiology (tissue and organismal metabolic status and function). Depending upon the radiation conditions and the desired endpoints, some properties will dominate over others as the major determinants of the final effects of the laser-tissue interactions.

For example, lasers emitting in the infrared domain of the electromagnetic spectrum interact with tissue with a photobiologic effect which is substantially photothermal. Photothermal effects result from the transformation of absorbed light energy to heat, leading to contraction, coagulation or destruction of the target tissue. The nature and extent of photothermal effects of the laser-tissue interactions are governed by (1) the distribution of light within the tissue, (2) tissue temperature, (3) duration of time the tissue is maintained at temperature, and (4) the tissue's thermal properties, diffusivity and heat capacity. These factors are collectively known as the “thermal history” of the tissue.

As the tissue temperature approaches the threshold temperature of vaporization of water (100° C.), the photothermal effects of the laser-tissue interactions come under (1) the influence of the energy requirements of the phase changes of the water, (2) tissue desiccation, (3) formation of steam vacuoles within the tissue, and (4) the mechanical effects of the rapidly expanding steam vacuoles trapped within the tissue.

The concept of an “effective optical absorption” as a function of depth is best represented by a Monte Carlo modeling calculation which includes the effects of initial light distribution striking the tissue (e.g., collimated light at normal incidence, diffuse light at non-normal incidence, etc.), the changes in the index of refraction at the air/tissue interface, absorption and scattering events within the tissue, and remittance from the tissue (by reflection at the air/tissue interface and by back scattering from within the tissue). Laser energy having a wavelength of between about 1.3 and 1.4 microns has an extinction depth of about 1.8 cm⁻¹. This wave length range is relatively poorly absorbed in water but by means of the photo-thermal mechanism associated with scattering will raise the temperature of the collagen core within the trabecular meshwork to the critical shrinkage temperature of 58 to 65 degrees Celsius.

Water strongly absorbs light at 2000 nanometers, leading to rapid vaporization of water. Tissue desiccation radically changes the optical characteristics of tissues, especially their absorption characteristics of infrared laser irradiation. In addition to the optical property changes, water loss reduces the thermal conductivity and specific heat of the tissue. Tissue “thermal history” is a dynamic function and must therefore be constantly monitored in order to attain the desired endpoint.

Pulsed Photothermal Radiometry

One method of monitoring the tissue thermal history has been derived from an understanding of the photothermal tissue effects of infrared lasers. This method, known as “pulsed photothermal radiometry” or PPTR, is a technique for determining tissue reaction with special reference to its thermal history. PPTR has been investigated as an indirect modality for the determining of the appropriate laser treatment for various tissues, such as skin, tendon and cornea. This procedure has not previously been used to modulate the thermal energy required for the shrinkage of the collagen connective tissue in the area of the ocular filtration mechanism.

Photothermal effects are produced within the target tissue when, by means of appropriate laser exposure parameters, the radiant energy exceeds the threshold required for tissue modification. The photothermal changes trigger a biological response which culminates in a complex sequence of events within the irradiated tissue. These changes may only be represented by a phase transition or may proceed to tissue destruction with a wound repair response and new tissue synthesis. In any case, the definitive change will be determined by the magnitude of the thermal response, or the “thermal history” of the tissue.

PPTR is a non-contact method that uses a rapid acting infrared detector to measure the temperature changes induced in a test material exposed to pulsed radiation. The heat generated as a result of light absorption by subsurface chromophores in the material diffuse to the surface and results in increased infrared emission levels at the surface. By collecting and concentrating the emitted radiation onto an infrared detector, one obtains a PPTR signal that represents the time evolution of temperature near the test material's surface. Useful information regarding the test material (e.g. cornea or skin tissue) may be deduced from the analysis of the PPTR signal, which might be used to modulate the coherent energy emitted. In this way, a closed loop feed back mechanism can be generated that will provide real-time intraoperative monitoring of the thermal energy required to shrink the target tissue.

Experiments have been conducted at the Beckman Laser Institute (University of California, Irvine) to determine the depth profiles of laser light absorption in skin tissue. It has been determined that strong scattering compared with absorption tends to raise the front surface temperature, as some of the scattered light is absorbed while back-scattering through the front surface. If the scattering is a significant event, the radiation transport, the temperature distribution, and the penetration depth are all dominated primarily by the scattering and not by the chromophore absorption. Transport through the sclera in the area of the trabecular meshwork will reveal a similar photothermal mechanism.

Colin Smithpeter, et al of the University of Texas, Austin, have shown that a continuous laser beam (CW) might be more efficacious than a pulsed emission in generating the appropriate thermal profile for collagen shrinkage. The thermal conduction of the CW laser operating at a similar wavelength over a longer period of time produces a deeper coagulation and a cone-shaped lesion. A sapphire lens contact probe reduces the beam divergence and the effective beam diameter. A smaller beam diameter increases the irradiance within the target site. The contact lens integrated into the probe also cools the corneal surface by conducting heat away from the epithelium thereby reducing the threat of superficial thermal damage.

In the case of treatment of the scleral spur site, the thermal profile without the contact lens or superficial heat-sink would be that of a long wedge profile. Conducting heat away from the surface would insure a maximal thermal modification of the tissue at the 800 micron depth of the trabecular meshwork. Physiologic temperature would be maintained in the more superficial corneal-scleral stroma and overlying conjunctival surface.

Brinkmann, et al of Lubeck, Germany, has investigated the influence of laser pulse energy and repetition rate. They showed that CW radiation, such as that emitted by a diode laser would lower the risk of tissue ablation due to the lack of peak intensities and peak radiant exposures, since the threshold needed for such damage would not be attained. The CW irradiation offers the possibility of achieving a spatial and temporal homogenous thermal profile.

The theoretical advantages of a CW emission is balanced by the benefits afforded by PPTR monitoring of the exposure parameters of a pulsed laser system.

Superficial Contact Cooling

It would be advantageous to conduct heat away from the front surface of the conjunctiva and sclera to assure the optimum thermal profile. This is defined as the maximal temperatures in the trabecular meshwork and near-normal temperatures in the more superficial tissues through which the laser energy has passed. Various thermal quenching devices might be postulated to provide this function.

It has been discovered that a combination of the appropriate application of pulsed laser irradiation and cryogen spray cooling may be used to protect the superficial tissues and confine the spatial distribution of thermal injury to the deeper target tissues.

A dynamic cooling process in accordance with the invention may be utilized by spraying the cryogen directly upon the site of laser application and permitting the surface cooling by means of evaporation. An example of the cryogen might be 1,1,1,2 tetrafluoroethane (R134a, cryogen's name in accordance with the National Institute of Standards and Technology; boiling point approximately −26 degrees Celsius). This cryogen is environmentally compatible, non-toxic, non-inflammable and will not damage the superficial ocular tissues.

A contact heat sink, either integrated within the laser contact delivery probe in the form of a passive static cooling system (quartz or sapphire contact surface through which the laser is delivered), or a separate corneo-scleral lens of the same materials would operate as a static heat sink because of its high thermal mass while permitting laser energy transmission.

Another embodiment of this cooling system might be a semi-dynamic system in which a cryogen spray is sprayed upon the lens or otherwise cools the lens before application to the eye.

Yet another embodiment of this cooling system might be a semi-dynamic system in which a cryogen solution is sprayed upon the lens or otherwise cools the lens before application to the eye. An appropriate method, which combines cooling and laser delivery, would be an integrated system utilizing the cooling element, a sapphire rod, as the distal part of the laser delivery system. The fiber optic delivery of the laser emission is interfaced with the sapphire rod with low energy losses. The sapphire rod would be hollowed out forming a cylinder at its distal end. The inside radius of the cylinder would be consistent with the inner limbal diameter. The outside diameters are actually shorter in the vertical and slightly smaller in the female, as well.

The cylinder will have a wall thickness of about 2 mm and will cover the circumferentially disposed target tissue, the scleral spur.

The distal end of the cylinder will have a hemispherical radius thus functioning as a cylindrical microlens system, which will act as a focusing element for the laser irradiation to the target site, the scleral spur.

This convex lens surface will contemporaneously function as the pressure-exerting element thus providing the local compression upon the sclera facilitating propagation of the laser beam.

In this manner the entire circumference of the target tissue will be superficially cooled, compressed and irradiated simultaneously to the depth of 600 to 800 u beneath the contact surface.

A variation of this principle might simplify the beam propagation. When the cylindrical cooling/delivery element is applied to the circum-limbal area after inserting the lid speculum, the angle of incidence of the laser beam will be less than 90 degrees, thus resulting in a longer optical pathway to the scleral spur. This will, however, be balanced by the fact that the laser beam pathway will be primarily through the homogeneous peripheral corneal tissue and only the most distal part of the pathway would be through turbid tissue. Exerting pressure upon the surface might not be necessary to further propagate the beam in this case.

An additional method of superficial cooling might be by means of thermal-electric means at the site of laser irradiation.

The invention having now been fully described, it should be understood that it may be embodied in other specific forms or variations without departing from its spirit or essential characteristics. Accordingly, the embodiments described above are to be considered in all respect as illustrative and not restrictive, the scope of the invention being indicated by the appended claims rather than the a foregoing description, and all changes, which come within the meaning and range of equivalency of the claims are intended to be embraced therein.

1. A method for the treatment of ocular collagen connective tissue comprising:
identifying a portion of the ocular collagen connective tissue having a connector portion which transitions into the ciliary muscle of an eye;
directing a source of energy at at least one selected site along the portion of the connective tissue, the amount of energy being sufficient to cause longitudinal shrinkage in the length of connective tissue.

2. A method as claimed in claim 1 wherein the energy source comprises a coherent light energy source for increasing the temperature of the connective tissue to produce a thermal phase transition thereof thereby causing the longitudinal shrinkage.

3. A method as claimed in claim 2 wherein coherent light energy source is an infrared laser.

4. A method as claimed in claim 2 wherein the increase in temperature of the connective tissue is controlled so that it falls within the range of the thermal shrinkage temperature of collagen (Tₛ).

5. A method as claimed in claim 4 wherein the thermal shrinkage temperature of collagen (Tₛ) is within about 5 degrees Celsius of about 23 degrees Celsius above ambient body temperature but below the temperature of coagulation and tissue destruction of the connective tissue.

6. A method as claimed in claim 1 wherein the amount of energy causing thermal shrinkage of the connective tissue is controlled so as to be atraumatic.

7. A method as claimed in claim 1 wherein the connective tissue site selected is one occupied by both tendinous insertion of the ciliary muscle and the trabecular meshwork of the aqueous filtration mechanism.

8. A method as claimed in claim 1 wherein the connective tissue selected for directing the source of energy is chosen so that the shrinkage opens the trabecular meshwork to increase the pore size and reduce resistance to aqueous outflow.

9. A method as claimed in claim 3 wherein the infrared laser emits light having a wavelength of about 1.32 microns with an extinction depth of about 800 to 1,000 microns.

10. A method as claimed in claim 3 wherein the infrared laser comprises a Neodymium:YAG laser operated at a repetition rate of from about 1 Hz to about 100 Hz.

11. A method as claimed in claim 1 wherein the source of energy is directed along a trans-scleral route to the connective tissue at the collagenous tendinous insertion of the ciliary muscle.

12. A method as claimed in claim 11 wherein the source of energy is directed to target the scleral spur.

13. A method as claimed in claim 1 wherein the energy is delivered by quartz fiber-optic probe having a diameter range of 200 to 320 micron, the probe being housed in a protective casing to provide a total outer diameter approximately equivalent to a 22 gauge needle.

14. A method as claimed in claim 13 further comprising the step of aiming a visible light beam along the probe to facilitate identification of the operative site.

15. A method as claimed in claim 1 further comprising the step of diagnostic gonioscopy to determine the filtration angle structure.

16. A method as claimed in claim 1 wherein the source of energy is applied in all four quadrants of the globe to shrink the connective tissue equally.

17. A method as claimed in claim 1 further comprising the step of staining the corneal and bulbar conjunctiva with fluorescein dye to facilitate delineation of the corneal-scleral trabecular meshwork.

18. A method as claimed in claim 10 wherein the Neodymium:YAG laser operates at about 300 microseconds pulse duration with a repetition rate of about 3 to 20 Hz and a power setting of about 1 to 6 watts.

19. A method as claimed in claim 18 wherein the laser has an energy per pulse of 6 Joules.

20. A method as claimed in claim 3 wherein the infrared laser comprises a 1.34 micron Neodymium:YAP laser operated so as to have an extinction depth near the depth of the target tissue.

21. A method as claimed in claim 14 wherein the aiming beam is a 0.5 mW Helium Neon 632.8 nm laser.

22. A method as claimed in claim 1 wherein the source of energy is selected from one or more of the following: microwave, radio frequency, ultrasound, sonic, electromagnetic, chemical or a combination of one or more thereof.

23. A method as claimed in claim 1 further comprising the step of applying a topical ophthalmic anesthetic.

24. A method as claimed in claim 1 further comprising the step of conducting heat away from the surface of the connective tissue.

25. A method as claimed in claim 24 wherein heat is removed from the surface by cryogen spray cooling.

26. A method as claimed in claim 24 wherein heat is conducted away from the surface of the connective tissue using passive cooling, dynamic cooling, or a combination thereof.

27. A method as claimed in claim 24 wherein heat is removed from the surface using a contact heat sink.

28. A method as claimed in claim 1 wherein the shrinkage in the connective tissue causes an increase in the functional mechanical advantage of the ciliary muscle to thereby increase the accommodative state of the lens of the eye.

29. A method as claimed in claim 1 wherein the shrinkage in the connective tissue causes a reduction of the resistance to aqueous outflow.

30. A system for the treatment of ocular collagen connective tissue comprising a probe, an energy source associated with the probe, the energy source being capable of providing thermal energy to cause an increase in temperature of the connective tissue to the thermal shrinkage temperature of collagen.

31. A system as claimed in claim 30 wherein the probe comprises a quartz fiber-optic probe having a diameter range of 200 to 320 micron, the probe being housed in a protective casing to provide a total outer diameter approximately equivalent to a 22 gauge needle.

32. A system as claimed in claim 30 further comprising a visible light beam located along the probe to facilitate identification of the operative site.

33. A system as claimed in claim 30 wherein the infrared laser comprises a Neodymium:YAG laser operating at about 300 microseconds pulse duration with a repetition rate of about 3 to 20 Hz and a power setting of about 1 to 6 watts.

34. A system as claimed in claim 30 wherein the laser has an energy per pulse of 6 Joules.

35. A system as claimed in claim 30 wherein the infrared laser comprises a 1.34 micron Neodymium:YAP laser operated so as to have an extinction depth near the depth of the target tissue.

36. A system as claimed in claim 32 wherein the Helium Neon laser beam is a 0.5 mW Helium Neon 632.8 nm laser.

37. A system as claimed in claim 30 wherein the energy source is one selected from following: infrared laser, microwave, radio frequency, ultrasound, sonic, electromagnetic, chemical or a combination of one or more thereof.

38. A system as claimed in claim 30 wherein heat is removed from the surface by cryogen spray cooling.

39. A system as claimed in claim 30 wherein heat is conducted away from the surface of the connective tissue using passive cooling, dynamic cooling, or a combination thereof.

40. A system as claimed in claim 38 wherein heat is removed from the surface using a contact heat sink.

41. A method for the treatment of presbyopia, the method comprising:
identifying a portion of ocular collagen connective tissue having a connector portion which transitions into the ciliary muscle of an eye;
directing a source of energy at at least one selected site along the portion of the connective tissue, the amount of energy being sufficient to cause longitudinal shrinkage in the length of connective tissue.

42. A method for increasing the mechanical advantage of a muscle, the method comprising:
identifying a portion of collagen connective tissue extending between the muscle and the base to which the connective tissue is attached;
directing a source of energy at at least one selected site along the portion of the connective tissue, the amount of energy being sufficient to cause longitudinal shrinkage in the length of connective tissue.

43. A method as claimed in claim 1 wherein the source of energy is directed to the portion of the ocular collagen connective tissue by means of a trans-scleral route directly to the said portion.

44. A method as claimed in claim 1 wherein the source of energy is directed to the portion of the ocular collagen connective tissue by directing the energy across the cornea and anterior chamber of the eye to a scleral spur ab interna.
